(19) United States
(12) Patent Application Publication
(10) Pub. No.: US 2005/0208555 A1
(43) Pub. Date: Sep. 22, 2005

(54) Methods of genotyping

(75) Inventor: Carl S. Raimond III, Pittsford, NY (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/083,251

(22) Filed: Mar. 16, 2005

Related U.S. Application Data
(60) Provisional application No. 60/553,935, filed on Mar. 16, 2004.

(51) Int. Cl. C12Q 1/68; C12P 19/34

(52) U.S. Cl. 435/6; 435/91.2

(57) ABSTRACT

Methods and kits are provided for genotyping a plurality of pre-selected SNPs. For each SNP a pair of oligos is synthesized. Each oligo is a perfect match to one allele of a polymorphic locus. The oligos are hybridized to genomic DNA and only those that are a perfect match to an allele that is present will hybridize. The hybridization reaction is size separated so that unbound oligos go with the small size fraction and bound oligos go with the large size fraction. The oligos in the large size fraction are amplified and detected to determine the genotype of the sample.

### RELATED APPLICATIONS

This application claims priority to U.S. Provisional Patent Application No. 60/553,935 filed Mar. 16, 2004, the disclosure of which is incorporated herein by reference in its entirety for all purposes.

### FIELD OF THE INVENTION

The methods of the invention relate generally to the fields of genetic analysis and genotyping.

### BACKGROUND OF THE INVENTION

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism.

### SUMMARY OF THE INVENTION

In one embodiment methods for genotyping polymorphisms, for example, single nucleotide polymorphisms are disclosed. In general allele specific oligonucleotides that are perfectly complementary to one allele of a SNP but have a mismatch at the polymorphic position for any other allele of the SNP are hybridized to the nucleic acid sample. If the allele is present the complementary ASO will bind to the target in the sample. Bound ASOs are separated from unbound ASOs by a size separation method and bound ASOs are amplified, for example, by PCR using common priming sites that flank the target specific region of the ASOs. If an allele is present the complementary ASO will be in the high molecular weight fraction and will be amplified. If an allele is not present the complementary ASP will be not be in the high molecular weight fraction and will not be amplified. The ASOs that are amplified can be detected, for example, by hybridization to an array that has probes complementary to each ASO. In one embodiment the ASOs are each tagged with a unique tag sequence so that amplified ASOs can be detected using a universal tag probe array. In another aspect the array comprises probes that are also allele specific and complementary to different variants of known polymorphisms that are complementary to ASOs in the assay.

In a preferred aspect the ASOs have a target complementary region flanked by common priming sites, allowing a single primer pair or a few primer pairs to be used to amplify the recovered ASOs. In some embodiments, the amplification product is labeled with a detectable label, for example, biotin.

The array of probes may be a plurality of probes that are complementary to the locus and allele specific regions of the oligonucleotides in the plurality of oligonucleotides.

In one embodiment, the oligonucleotides in the plurality of oligonucleotides further comprise a tag and each pair of oligonucleotides comprises the same tag sequence which is different from the tag sequence in every other pair of oligonucleotides. An array of tag probes that are complementary to the tags present in the plurality of oligonucleotides may be used to detect the presence or absence of specific alleles in the sample. In one embodiment each oligonucleotide is labeled with a different tag.

In one embodiment the oligonucleotides further comprise a first restriction site between the 5′ common priming site and the locus and allele specific region and a second restriction site between the 3′ common priming site and the locus and allele specific region. Following amplification the amplification product may be digested with a first restriction enzyme that recognizes the first restriction site and a second restriction enzyme that recognizes the second restriction site. The first and second restriction enzymes may be the same.

### BRIEF DESCRIPTION OF THE FIGURE

FIG. 1 shows a schematic of one embodiment of the invention. Allele specific oligonucleotides complementary to selected polymorphisms are mixed with the genomic sample to be analyzed. The sample is fractionated to remove oligonucleotides that are not stably associated with the genomic DNA. Those oligonucleotides that are associated with the genomic DNA are amplified and detected to determine the genotype of the polymorphisms.

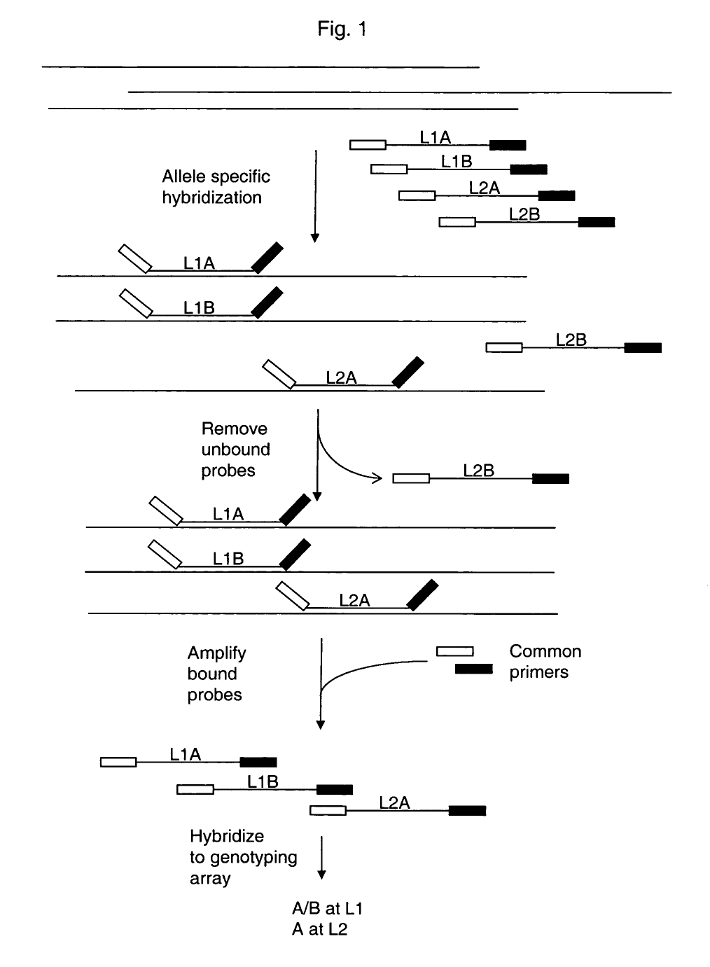

### DETAILED DESCRIPTION OF THE INVENTION

### A) GENERAL

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being, but may also include other organisms including but not limited to mammals, plants, fungi, bacteria or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. patent application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, New York, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and 6,107,023 and U.S. Ser. Nos. 09/916,135, 09/920,491 (U.S. patent application Publication 20030096235), U.S. Ser. No. 09/910,292 (U.S. patent application Publication 20030082543), and U.S. Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO 99/47964), each of which also is hereby incorporated by reference in its entirety for all purposes. Instruments and software may also be purchased commercially from various sources, including Affymetrix.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication No. 20020183936), U.S. Ser. No. 10/065,856, 10/065,868, 10/328,818, 10/328,872, 10/423,403, and 60/482,389.

### B) DEFINITIONS

“Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded or entirely single stranded. A double stranded adaptor may comprise two oligonucleotides that are at least partially complementary. The adaptor may be phosphorylated or unphosphorylated on one or both strands.

Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with Pvull the blunt ends can be converted to a two base pair overhang by incubating the fragments with Klenow in the presence of dTTP and dCTP. Overhangs may also be converted to blunt ends by filling in an overhang or removing an overhang.

Methods of ligation will be known to those of skill in the art and are described, for example in Sambrook et at. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E.coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′→5′ phosphodiester bond, substrates include single-stranded RNA and DNA as well as dinucleoside pyrophosphates; or any other methods described in the art. Fragmented DNA may be treated with one or more enzymes, for example, an endonuclease, prior to ligation of adaptors to one or both ends to facilitate ligation by generating ends that are compatible with ligation.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “epigenetic” as used herein refers to factors other than the primary sequence of the genome that affect the development or function of an organism, they can affect the phenotype of an organism without changing the genotype. Epigenetic factors include modifications in gene expression that are controlled by heritable but potentially reversible changes in DNA methylation and chromatin structure. Methylation patterns are known to correlate with gene expression and in general highly methylated sequences are poorly expressed.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” Hybridizations are usually performed under stringent conditions, for example, at a salt concentration of no more than about 1 M and a temperature of at least 25° C. For example, conditions of 5× SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004, available at Affymetrix.com.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” as used herein refers to an intentionally created collection of nucleic acids which can be prepared either synthetically or biosynthetically and screened for biological activity in a variety of different formats (for example, libraries of soluble molecules; and libraries of oligos tethered to beads, chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 5%, 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

Genotyping by Hybridization of Locus and Allele Specific Oligonucleotides

Methods of genotyping polymorphisms are disclosed. Generally the methods involve hybridizing allele specific oligonucleotides (ASOs) to genomic DNA in solution, separation of the bound ASOs from ASOs that are not bound to the genomic DNA and detection of the bound ASOs, preferably by hybridization to an array of probes that are complementary to the ASOs. In a preferred aspect, the ASOs are locus and allele specific so the presence of an ASO in the bound fraction is indicative of the presence of a selected allele in the genomic sample. Many polymorphisms may be genotyped in parallel, for example, more than 100, 1000, 10,000, 100,000 or 1,000,000 polymorphisms may be genotyped in a single experiment using the methods. The polymorphisms may be, for example, single nucleotide polymorphisms (SNPs) or an insertion or deletion or one or more bases.

In one embodiment, SNPs are genotyped. For each SNP to be genotyped two oligos are used: one that is perfectly complementary to the A allele and one that is complementary to the B allele. In preferred aspects the two ASOs for any given SNP are identical except for the position that is complementary to the polymorphic position. The position of the polymorphic position within the ASO is preferably within 5 bases of the center of the ASO. In a preferred embodiment the oligonucleotide comprises 25 nucleotides that are complementary to the SNP and the region surrounding the SNP. The 13^{th} position of the 25 is complementary to one allele of the SNP and the positions 1-12 and 14-25 are complementary to the 12 bases that are immediately 3′ of the SNP and the 12 bases that are immediately 5′ of the SNP. In one embodiment the oligonucleotides further comprise 5′ and 3′ regions that are common to a plurality of the oligonucleotides and may be used as universal priming sites so that a single PCR reaction with a single primer or pair of primers may be used to amplify the ASOs. For example, the construction of the oligonucleotides may be: 5′-first priming site-allele specific oligonucleotide-second priming site-3′. The first and second priming sites are preferably not self complementary.

A plurality of ASOs may be synthesized and combined. For example, a plurality of 100 or more, 1,000 or more, 10,000 or more or 100,000 or more SNPs may be selected. The SNPs may be of interest, for example, because they are in a region or regions of interest, because they are spaced throughout a region of the genome or a genome or because they are thought to have an association with a particular phenotype or phenotypes. For each SNP to be genotyped an ASO is designed that is a perfect match to each variant so for a SNP that has two alleles A and B one ASO is designed to be complementary to allele A and a second ASO is designed to be complementary to allele B. The oligonucleotides may be mixed together and added as a pool to a sample containing nucleic acid to be genotyped.

The oligonucleotides are hybridized to a nucleic acid sample so that the oligonucleotides hybridize specifically to nucleic acids in the sample that are the exact complement of the interrogation region of the oligonucleotides over the length of the interrogation region. The perfect match probe for an allele should hybridize to that allele and not to the alternate allele of that SNP. The oligonucleotide for allele A should hybridize to nucleic acids containing allele A but not allele B and likewise the oligonucleotide that is complementary to allele B should hybridize to nucleic acids containing allele B but not allele A. The nucleic acid sample may be genomic DNA, mRNA, total nucleic acid, total RNA or an amplification product, for example, a product of multiple displacement amplification (MDA) of a genomic sample. The sample may be fragmented prior to hybridization of the ASO mixture. Fragmentation may be random, for example, by shearing, sonication or DNAse treatment or it may be sequence specific, for example, by restriction endonuclease digestion. In preferred aspects the sample is denatured, for example, by heating.

Following hybridization unbound ASOs are separated from bound ASOs. The separation may be by, for example, size separation. In one embodiment, the hybridization mix is run through a size exclusion column and the high molecular weight fraction is separated from the low molecular weight fraction. Unbound ASOs should be present in the low molecular weight fraction. The bound oligonucleotides should be bound to nucleic acids that are greater than 500 base pairs or greater than 1,000 base pairs and the unbound oligonucleotides are small, for example, less than about 200 bases and most preferably less than about 100 bases. Size separation may be accomplished by, for example, size exclusion chromatography or non-denaturing gel electrophoresis. Those of skill in the art are familiar with methods of separating large complexes from small fragments or oligonucleotides. In one aspect gel-filtration chromatography methods may be used. In gel filtration chromatography a stationary phase consisting of porous beads with a defined range of pore sizes is used. Molecules that are small enough to fit into the pores elute most slowly while molecules that are too big to fit into the pores stay in the mobile phase between the beads and elute first. The resin can be selected to have pore size or physical characteristics that provide for optimal separation of the unbound ASOs and the ASOs bound to nucleic acids in the sample. Many different types of resin are available.

The ASOs are mixed with the sample under conditions that allow allele specific hybridization of the ASOs to their complementary target. The hybridization conditions should be sufficiently stringent so that the ASO binds stably only to the allele that it is complementary to and not to the alternate allele, even though the difference may be only a single base. If an allele is absent the ASO that is complementary to that allele should not bind to a target in the sample. The fraction containing the target bound ASOs, the “high molecular weight fraction”, may then be analyzed to determine which of the oligonucleotides from the plurality are present. In one embodiment, the oligonucleotides present in the larger fraction are amplified using common priming sites present in the ASOs. In a preferred embodiment the amplification product is labeled either during amplification by incorporation of labeled nucleotides or by end labeling the amplification products using, for example, terminal transferase.

The amplification product may be hybridized to an array to detect which alleles of the SNPs are present in the starting sample. In a preferred aspect the array comprises probes that are perfectly complementary to the locus and allele specific portion of the ASOs. If the SNP is homozygous, only one of the ASOs should be amplified from the larger fraction and detected by hybridization. If the SNP is heterozygous, both ASOs should be amplified and detected by hybridization. The array includes individual features that contain probes that are allele specific and complementary to the ASOs.

The array may comprise allele specific probes for a plurality of pre-selected SNPs and in a preferred embodiment the array is designed to detect the oligonucleotides in the plurality of SNPs selected by one or more researchers. Panels of ASOs may be designed to genotype panels of 100, 300, 500, 1,000, 3,000, 5,000 or 10,000 or more SNPs of interest. A plurality of SNPs of interest may be selected, for example for a specific research interest, pairs of oligos may be designed and synthesized to be used to assay the genotype of those SNPs using the methods disclosed and an array may be designed and synthesized to detect the SNPs in the plurality. The array may be customized to interrogate individual pools of SNPs selected by a researcher. The array may be designed with 1 or more probes that are perfectly complementary to one allele of a polymorphic position and not to another allele of that polymorphic position so that hybridization is allele specific under selected hybridization conditions. In one embodiment features may be present in duplicate or triplicate, so that the same probe is present in different positions on the array. In some embodiments mismatch probes are included for one or more alleles of one or more of the SNPs to be analyzed.

In another embodiment the ASOs comprise a tag sequence that is allele specific. The tag may be, for example 20, 21-25 or 25-30 nucleotides in length. Tags and specific sets of tag and tag probe sequences are disclosed in U.S. Pat. No. 6,458,530 and U.S. patent application Ser. No. 09/827,383, each of which is incorporated herein by reference in its entirety. In general, tag sequences are selected that are not present in the genome of interest so they do not cross hybridize with the genome. Tags are also generally used in sets of, for example, 100 to 10,000 and tags of the set are selected so that they do not cross hybridize with another tag in the set or with the complement of another tag in the set. Each allele of each SNP may be tagged with a different tag sequence so that the presence or absence of an oligo in the large fraction amplification product may be analyzed by hybridization to an array of tag probes. The tag is complementary to the tag probe and will hybridize specifically to the feature on the array containing the tag probe. Each feature of the array corresponds to a single tag probe sequence so that the probes in that feature are primarily of the sequence of a single tag probe, although due to manufacturing processes many of the individual probes in any given feature may be shorter than the full length tag probe (truncated versions of the full length) due to incomplete synthesis. In one embodiment a tag probe array such as GenFlex, available from Affymetrix, Santa Clara, is used to detect the presence of tags in the amplified mixture.

In some embodiments the universal primers are removed prior to labeling or prior to hybridization by, for example, digesting with a restriction enzyme at a restriction enzyme site that was incorporated into the oligonucleotides. This may allow for improved efficiency and specificity of hybridization.

In one embodiment the oligos for each allele of a biallelic SNP may have different universal oligonucleotides incorporated. For example, the A allele oligo may have universal primer set 1 flanking the locus and allele specific region and the B allele oligo may have universal primer set 2 flanking the locus and allele specific region. Allele A could then be amplified with universal primer set 1 and allele B could be amplified with universal primer set 2. Parallel amplification reactions could be used, one with universal primer set 1 and one with universal primer set 2 and differentially detectable labels could be incorporated into the different reactions. In another embodiment the primers could be differentially labeled and both primer sets could be used for amplification in a single reaction. In another embodiment the A and the B allele could be tagged with the same tag and differentially amplified using the two primer sets. This would allow for differential detection of both alleles in the same tag array hybridization experiment. In another aspect, the different alleles may be differentially labeled by incorporation of a specific sequence and detection may be by hybridization of a labeled oligonucleotide that is complementary to the sequence. This type of sandwich hybridization assay may be used to label subsets of the ASOs with different labels. In some aspects 2, 3, 4 or more different labels may be used. The different alleles may be labeled differently or different subsets of polymorphisms may be labeled with different labels. For polymorphisms that have more than 2 alleles each allele may be labeled with a differentially detectable label.

In some embodiments kits to perform the methods are contemplated. The kit may comprise a collection of oligos that are complementary to different alleles for a plurality of pre-selected polymorphisms. In a preferred aspect the polymorphisms are SNPs. Sets of ASOs may be made for collections of SNPs of interest. In some aspects more the kit includes ASOs for more than 100, 200, 300, 500, 1,000, 1,500, 3,000 or 10,000 different SNPs. The kit may also comprise arrays, which may be allele and locus specific or arrays of tag probes, for example, the Affymetrix GENFLEX® tag probe array.

### Comclusion

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

1. A method for genotyping a plurality of polymorphisms in a nucleic acid sample comprising:
(a) hybridizing a plurality of allele specific oligonucleotides to the nucleic acid sample, wherein each allele specific oligonucleotide is perfectly complementary to a genomic region including a polymorphism;
(b) fractionating the nucleic acid sample from (a) to separate high molecular weight nucleic acids from low molecular weight nucleic acids, wherein the high molecular weight nucleic acids are included in a high molecular weight fraction;
(c) amplifying allele specific oligonucleotides in the high molecular weight fraction;
(d) hybridizing the amplification product from (c) to an array of probes to obtain a hybridization pattern; and
(e) determining the genotype of the nucleic acid sample for at least some of the plurality of polymorphisms by analyzing the hybridization pattern.

2. The method of claim 1 wherein each oligonucleotide in the plurality of allele specific oligonucleotides comprises a 5′ common priming site, common to a plurality of the oligonucleotides, and a 3′ common priming site, common to a plurality of the oligonucleotides, and an allele specific region that is unique to each oligonucleotide in the plurality.

3. The method of claim 2 wherein each oligonucleotide in the plurality of allele specific oligonucleotides further comprises a tag sequence that is unique to each oligonucleotide in the plurality.

4. The method of claim 2 wherein the allele specific oligonucleotides in the high molecular weight fraction are amplified by polymerase chain reaction using primers complementary to the 5′ common priming site and the 3′ common priming site.

5. The method of claim 1 wherein the amplification product is labeled with a detectable label.

6. The method of claim 5 wherein the detectable label is biotin.

7. The method of claim 2 wherein a plurality of the polymorphisms are single nucleotide polymorphisms.

8. The method of claim 7 wherein the plurality of allele specific oligonucleotides comprises a different allele specific oligonucleotide for each allele of each single nucleotide polymorphism.

9. The method of claim 2 wherein the plurality of allele specific oligonucleotides comprises a first oligonucleotide and a second oligonucleotide for each polymorphism to be genotyped, wherein the first oligonucleotide is complementary to a first allele of the polymorphism and the second oligonucleotide is complementary to a second allele of the polymorphism.

10. The method of claim 9 wherein the first oligonucleotide and the second oligonucleotide further comprise a tag sequence that is different for each polymorphism to be genotyped and the array comprises probes complementary to the tag sequences.

11. The method of claim 3 wherein the array of probes comprises an array of tag probes that are complementary to the tags present in the plurality of oligonucleotides.

12. The method of claim 2 wherein the oligonucleotides further comprise a first restriction site between the 5′ common priming site and the allele specific region and a second restriction site between the 3′ common priming site and the locus and allele specific region.

13. The method of claim 12 wherein the amplification product is digested with a first restriction enzyme that recognizes the first restriction site and a second restriction enzyme that recognizes the second restriction site.

14. The method of claim 1 wherein the array comprises allele specific probes complementary to the plurality of allele specific oligonucleotides.

15. A method for detecting the presence of a plurality of selected target sequences in a nucleic acid sample comprising:
mixing the nucleic acid sample with a plurality of probes under conditions to allow complex formation between probes and targets, wherein the probes comprise a central region comprising at least 20 bases that are perfectly complementary to a unique target sequence, a first common priming site 5′ of the central region and a second common priming site 3′ of the central region, to generate probe:target complexes;
separating probe:target complexes from probe that is not in a complex with a target;
amplifying the probe in the probe:target complexes by polymerase chain reaction with a first primer to the first common priming site and a second primer to the second common priming site; and,
detecting the amplified probes by hybridization to an array.

16. The method of claim 15 wherein the targets are each one allele of a polymorphism from a plurality of polymorphisms.

17. A kit comprising a plurality of allele specific oligonucleotide comprising oligonucleotides complementary to each variant of each of at least 500 human single nucleotide polymorphisms, wherein each allele specific oligonucleotide comprises a region of at least 19 bases that is perfectly complementary to the variant position of a single nucleotide polymorphism and the 9 bases immediately upstream and the 9 bases immediately downstream of the variant position of said single nucleotide polymorphism and, wherein each allele specific oligonucleotide further comprises a first common priming site and a second common priming site so that all of the allele specific oligonucleotides in the plurality may be amplified using the same pair of primers.

18. The kit of claim 17 wherein each allele specific oligonucleotide further comprises an identifying tag sequence that is different for each polymorphic position.

19. The kit of claim 17 wherein each allele specific oligonucleotide further comprises an identifying tag sequence that is different for each oligonucleotide.

20. The kit of claim 17 further comprising an array comprising probes complementary to the allele specific region of each allele specific oligonucleotide.
